(19) United States
(12) Patent Application Publication
(10) Pub. No.: US 2012/0095239 A1
(43) Pub. Date: Apr. 19, 2012

(54) A PROCESS FOR THE PREPARATION OF DULOXETINE HYDROCHLORIDE

(75) Inventors: Ravi Ponnaiah, Vadodara (IN); Ashok Prasad, Vadodara (IN); Killol Patel, Vadodara (IN); Hitarth Harshendu Acharya, Vadodara (IN)

(73) Assignee: ALEMBIC PHARMACEUTICALS LIMITED, Vadodara, Gujarat (IN)

(21) Appl. No.: 13/256,361

(22) PCT Filed: Mar. 5, 2010
(86) PCT No.: PCT/IB10/50957

§ 371 Date: Dec. 5, 2011

(30) Foreign Application Priority Data
Mar. 13, 2009 (IN) 1176/MUM/2009

(51) Int. Cl. C07D 333/20

(52) U.S. Cl. 549/75

(57) ABSTRACT

The present invention relates to a process for the preparation of Duloxetine hydrochloride of formula (I).

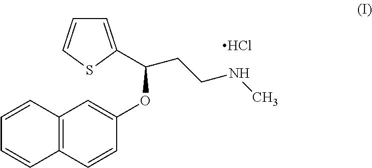

### FIELD OF THE INVENTION

The present invention relates to a process for the preparation of Duloxetine hydrochloride of formula (I).

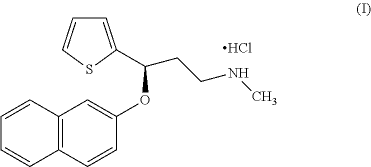

### BACKGROUND OF THE INVENTION

Duloxetine chemically known as (+)-(5)-N-methyl-γ-(1-naphthyloxy)-2-thiophenepropyl amine belongs to class of antidepressant. Duloxetine is a serotonin-norepinephrine reuptake inhibitor (SNRI) used for major depressive disorder (MDD), generalized anxiety disorder (GAD), pain related to diabetic neuropathy and fibromyalgia Its hydrochloride salt is marketed under brand name of Cymbalta®.

Duloxetine and its pharmaceutically acceptable salt were first disclosed in U.S. Pat. No. 5,023,269. The process for synthesis of Racemic Duloxetine as described in this patent is as follows:

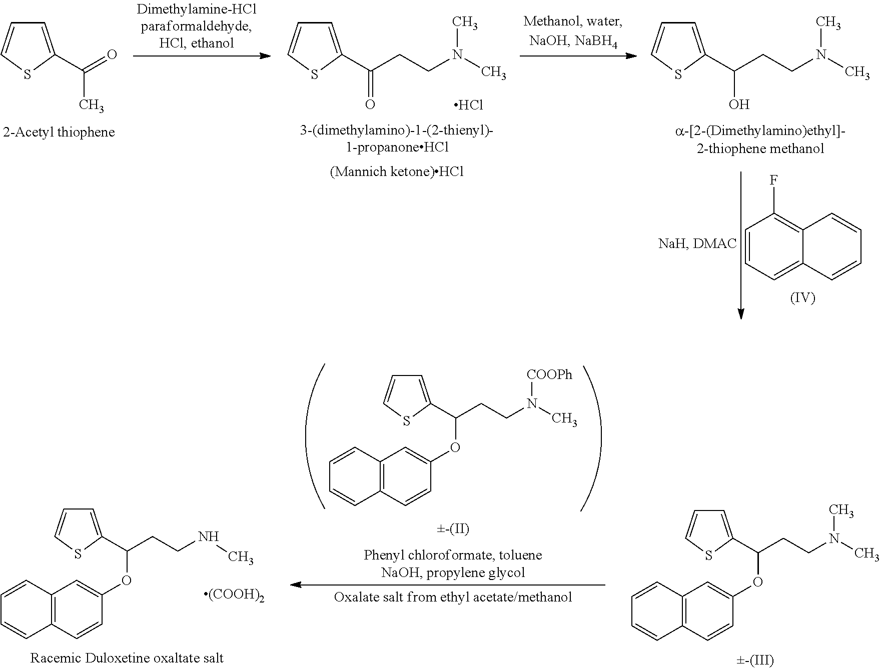

This patent teaches recrystalization of racemic Duloxetine oxalate from ethyl acetate/methanol. The process from crystallization of Duloxetine hydrochloride is not exemplified or disclosed in the specification.

EP 457559 and Tetrahedron Letters, Vol. 31, No. 49, pp7101-7104, 1990 describe process for the preparation of Duloxetine base by converting it to oxalate salt in ethyl acetate as solvent.

U.S. Pat. No. 5,362,886 depicts that after hydrolysis of carbamate intermediate using DMSO/NaOH, the reaction mixture was acidified to pH 5.0-5.5 using acetic acid which leads to formation of Duloxetine acetate salt. Further, hexane is added to the reaction mixture and layers are separated. The aqueous layer is then basified to pH 10.5 using NaOH and extracted with ethyl acetate. The combined organic layers are washed with water and concentrated. To concentrated organic layers conc. HCl is added followed by seeding to obtain Duloxetine hydrochloride. By following this process for preparation of Duloxetine hydrochloride it is found that 4-Napthol impurity of formula (A) is present in final compound more than 0.3% and total impurity greater than 0.55%.

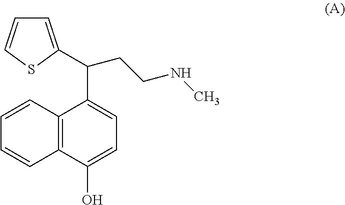

The inventors of present invention have serendipitously discovered that if preparation of Duloxetine hydrochloride is carried out in the presence of acetone and ethyl acetate-HCl, the 4-Napthol impurity of formula (A) is found to be not more than 0.01% and total impurity not more than 0.06%. Also, the quality of finished product is improved. Thereby the process is rendered cost effective and economically viable.

The ICH Q3A(R1) guidance for API manufacturers requires that process impurities should be maintained below set limits by controlling process parameters, such as temperature, pressure, time, and stoichiometric ratios, and including purification steps, such as crystallization, distillation, and liquid-liquid extraction, in the manufacturing process. Therefore, the process of present invention offers advantage over prior art process since the process impurities are reduced by significant ratio.

### OBJECTS OF THE INVENTION

It is the primary object of the present invention to provide a process for the preparation of Duloxetine hydrochloride of formula (I).

Another object of the present invention is to provide a process for the preparation of Form A of Duloxetine hydrochloride.

### SUMMARY OF THE INVENTION

The present invention provides a process for the preparation of Duloxetine hydrochloride of formula (I).

An aspect of present invention provides a process for the preparation of Duloxetine hydrochloride of formula (I) comprising of converting Duloxetine base to Duloxetine hydrochloride characterized in that the said conversion is carried out in acetone and ethyl acetate-HCl.

Further aspect of the present invention provides a process for the preparation of Duloxetine hydrochloride comprising steps of:
(a) treating salts of Duloxetine with base to obtain Duloxetine base
(b) converting Duloxetine base to Duloxetine hydrochloride characterized in that the said conversion is carried out in acetone and ethyl acetate-HCl.

Yet another aspect of the present invention provides a process for the preparation of Duloxetine hydrochloride comprising steps of:
(a′) treating salts of Duloxetine with base to obtain Duloxetine base
(b′) dissolving duloxetine base in acetone
(c′) treating said solution in step (b) with ethyl acetate-HCl .to obtain Duloxetine hydrochloride

Further aspect of the present invention provides a process for the preparation of Form A of Duloxetine hydrochloride comprising steps of:
(a) treating salts of Duloxetine with base to obtain Duloxetine base
(b) converting Duloxetine base to Duloxetine hydrochloride characterized in that the said conversion is carried out in acetone and ethyl acetate-HCl
(c) isolating form A.

### BRIEF DESCRIPTION OF THE DRAWINGS

FIG. 1: PXRD of Duloxetine hydrochloride Form A (obtained by Example-2)

### DETAILED DESCRIPTION OF THE INVENTION

A preferred embodiment of the present invention provides a process for the preparation of Duloxetine hydrochloride of formula (I) comprising of converting Duloxetine base to Duloxetine hydrochloride characterized in that the said conversion is carried out in acetone and ethyl acetate-HCl.

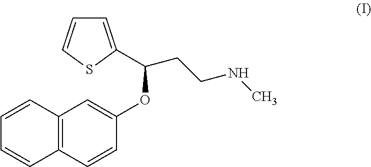

An embodiment of the present invention provides a process for the preparation of Duloxetine hydrochloride comprising steps of:
(a) treating salts of Duloxetine with base to obtain Duloxetine base
(b) converting Duloxetine base to Duloxetine hydrochloride characterized in that the said conversion is carried out in acetone and ethyl acetate-HCl.

Another embodiment of the present invention provides a process for the preparation of Duloxetine hydrochloride comprising steps of:
(a′) treating salts of Duloxetine with base to obtain Duloxetine base
(b′) dissolving duloxetine base in acetone
(c′) treating said solution in step (b) with ethyl acetate-HCl.to obtain Duloxetine hydrochloride

Said salts of Duloxetine includes but are not limited to organic and inorganic acid salts for example, hydrochloric, hydrobromic, sulfuric, phosphoric, para-toluenesulfonic, methanesulfonic, oxalic, maleic, acetic acid and the like.

The salts of Duloxetine are treated with aqueous solution of base such as alkali and alkaline earth metal hydroxide, carbonate and bicarbonate, for example sodium hydroxide, potassium hydroxide, calcium hydroxide, sodium carbonate, potassium carbonate, sodium bicarbonate and the like. The treatment can be carried out in the presence of suitable organic solvent like ethyl acetate, toluene and the like and the organic layer can be separated. Alternatively, the aqueous reaction mixture can be extracted with suitable organic solvent like ethyl acetate, toluene and the like. The organic layer thus obtained can be concentrated to obtain Duloxetine base of formula (I) in form of oily residue.

Further, the Duloxetine base is dissolved in acetone at about ambient temperature or elevated temperature to obtain a solution. The solution thus obtained can be optionally filtered through hyflo bed. Alternatively the solution can be treated charcoal or sodium dithionate and filtered by conventional methods.

The filtrate thus obtained is treated with ethyl acetate-HCl to adjust pH at about 1 to 2. Since, Duloxetine hydrochloride is insoluble in acetone as well as ethyl acetate, the hydrochloride salt gets precipitated out which is then isolated by conventional procedures like filtration or centrifugation.

Ethyl acetate-HCl is prepared by purging hydrochloride gas in ethyl acetate to obtain a saturated solution.

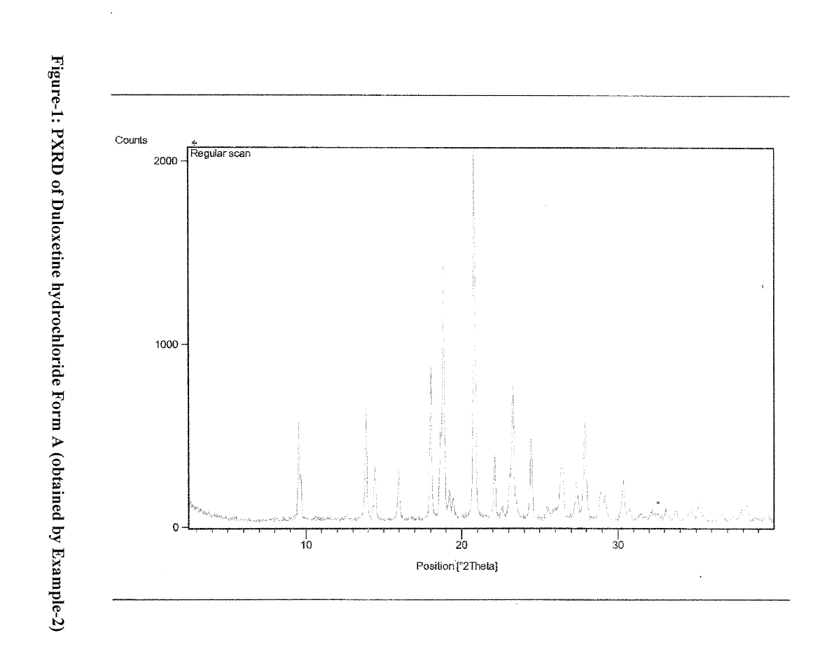

The XRD of Duloxetine hydrochloride thus obtained matches with Form A as shown in FIG. 1 which is prior art form.

Yet another embodiment of the present invention provides a process for the preparation of Form A of Duloxetine hydrochloride comprising steps of:
(a) treating salts of Duloxetine with base to obtain Duloxetine base
(b) converting Duloxetine base to Duloxetine hydrochloride characterized in that the said conversion is carried out in acetone and ethyl acetate-HCl
(c) isolating form A.

The following examples illustrate the invention further. It should be understood however, that the invention is not confined to the specific limitations set forth in the individual example but rather to the scope of the appended claims.

### Example-1

### Preparation of Duloxetine base

Charge D M Water (500 ml) into a RBF. Charge Duloxetine hydrochloride (100 g) to the flask. Charge Ethyl acetate (500 ml) to the flask. Stir the reaction mixture for 10-15 min. 25-35° C. Add 10% aq. Sod. Hydroxide solution (68 ml) to the flask maintaining temperature between 10-15° C. and adjust pH of the reaction mass to 9-10. Stir the reaction mixture for 10-15 min. at 10-15° C. Separate the org. layer. Re-extract the aq. layer with Ethyl acetate (500 ml). Combine both org. layer and wash with D M Water (500 ml). Recover ethyl acetate completely from org. layer at below 50° C. under vacuum to obtain oily residue

### Example-2

### Preparation of Duloxetine hydrochloride

Charge Acetone (900 ml) to the Duloxetine base obtained in Example-1 at 25-35° C. Stir the reaction mixture for 10-15 mins. Filter the reaction mass through to hyflo. Wash hyflo bed with acetone (100 ml). Transfer the filterate to another RBF. Add Ethyl acetate HCl (150 ml) to the reaction mixture at 25-35° C. to adjust the pH˜1. Stir the reaction mixture for 2 hrs. at 25-35° C. Filter the content. Wash the wet cake with Acetone (100 ml). Suck dry the wet cake for 30-45 mins. Dry the material under vacuum at 40-50° C. till LOD comes below 0.5%.
Yeild: ˜95% (w/w)
Purity: 99.94%
Impurity (A): 0.01%
Total Impurity: 0.06%
   PXRD: FIG. 1 (Form A)

1. A process for the preparation of Duloxetine hydrochloride comprising of converting Duloxetine base to Duloxetine hydrochloride characterized in that the said conversion is carried out in acetone and ethyl acetate-HCl.

2. A process for the preparation of Duloxetine hydrochloride comprising steps of:
(a) treating salts of Duloxetine with base to obtain Duloxetine base
(b) converting Duloxetine base to Duloxetine hydrochloride characterized in that the said conversion is carried out in acetone and ethyl acetate-HCl.

3. A process according to claim 2, comprising steps of:
(a′) treating salts of Duloxetine with base to obtain Duloxetine base
(b′) dissolving duloxetine base in acetone
(c′) treating said solution in step (b) with ethyl acetate-HCl to obtain Duloxetine hydrochloride.

4. A process according to claims 2, wherein said salts of Duloxetine of formula (I) comprises of hydrochloric, hydrobromic, sulfuric, phosphoric, paratoluenesulfonic, methanesulfonic, oxalic, maleic and acetic acid.

5. A process according to claim 2, wherein said base comprises of sodium hydroxide, potassium hydroxide, calcium hydroxide, sodium carbonate, potassium carbonate and sodium bicarbonate.

6. A process according to claim 1, wherein said Duloxetine hydrochloride is obtained in polymorphic Form A.

7. A process according to claim 6, wherein process for preparation of form A comprises steps of:
(a) treating salts of Duloxetine with base to obtain Duloxetine base
(b) converting Duloxetine base to Duloxetine hydrochloride characterized in that the said conversion is carried out in acetone and ethyl acetate-HCl
(c) isolating form A
